(19) United States
(12) Patent Application Publication
(10) Pub. No.: US 2003/0195448 A1
(43) Pub. Date: Oct. 16, 2003

(54) Heat regulating wound dressing

(76) Inventor: Jeffrey L. Jensen, (US)

Correspondence Address: Glenn K. Beaton, Suite 4100, 1801 California Street, Denver, CO, 80202, US

(21) Appl. No.: 10/123,369

(22) Filed: Apr. 16, 2002

(51) Int. Cl. A61F 13/00; A61F 5/00

(52) U.S. Cl. 602/41; 602/2

(57) ABSTRACT

A wound dressing having exothermic materials that undergo a chemical reaction to generate heat to produce a desired temperature to promote healing at a wound site, and a phase-change material having a phase-change temperature to absorb heat and maintain the wound dressing at said desired temperature.

### FIELD OF THE INVENTION

The present invention relates to the field of dressings for soft tissue wounds and, in particular, a dressing that incorporates a phase-change material, i.e., a material that produces or absorbs substantial amounts of heat as it passes between liquid, solid and gaseous phases, in order to maintain a constant desired wound temperature to promote tissue growth and healing.

### BACKGROUND OF THE INVENTION

Tissue wounds are common injuries. Wounds can be internal or external. Discontinuity in the integrity of the skin is typically referred to as an external wound.

External wounds can be produced from external trauma, such as impact or cutting forces. Such external trauma may be deliberate, as in the case of an incision produced by a surgeon's scalpel, or accidental as in the case of scrapes and cuts produced by common accidents. A further category of external wounds is chronic wounds produced in a predictable but to some extent unavoidable manner by chronic friction, pressure and inflammation, e.g., diabetic foot ulcers, pressure ulcerations (bed sores) and venous insufficiency ulcerations.

The formation and healing of external chronic wounds is aggravated by vascular disease. Reductions in blood flow to a tissue site can itself produce a wound by resulting in insufficient tissue oxygenation or insufficient fluid drainage. Because vascular disease is typically chronic, the resultant wounds are also chronic.

Diabetes presents special wound complications and severity. Diabetes patients often suffer from varying degrees of vascular disease and sensory peripheral neuropathy; in other words, their feet and hands tend to be insensitive. In more extreme cases, the patient has little or no sensation in his feet. When a wound develops in such a patient, the patient experiences no pain, and indeed may be wholly unaware of the wound. Many of these wounds are on pressure points on the foot. As the insensitive patient continues to apply pressure to these wound points, as by simply walking, the wound becomes very severe, ulcerated and perhaps infected. The repeated trauma of ambulation contributes to the wound formation at the outset. Vascular impairment further compromises the healing of these wounds. It has been estimated that there are 800,000 diabetic foot ulcer cases per year in the United States. The treatment cost for these is over $5,000 per year per patient, for an annual cumulative cost of about $5 billion per year.

Much of this treatment is ultimately unsuccessful, and when the wound becomes infected often the only option is amputation to save the patient's life. These diabetic foot ulcers result in about 67,000 foot amputations per year in the United States alone. They account for more hospitalizations than any other single complication of diabetes. The problem appears to be worsening; the lower extremity amputation rate has increased each year since 1990. About 84% of these amputations are preceded by foot ulcers.

Diabetic foot ulcers present challenges that are quite specific in the field of chronic wound care, and which vary widely. For example, forefoot wounds are generally considered low exudate wounds, i.e., they produce little fluid to the wound surface. For that reason, they do not require absorptive dressings, and sometimes do require moisture-giving gels to maintain sufficient humidity at the wound site to promote healing. Conversely, midfoot and rearfoot wounds associated with Charcot deformity are much larger and produce much more exudate. They require a highly absorptive dressing that removes exudate from the wound site to reduce the possibility of maceration and damaging the surrounding skin.

The levels of wound care dressing choices are explained well in the book Chronic Wound Care:

The following are some general rules that apply when choosing a dressing: (1) use a dressing that will keep the ulcer bed continuously moist; (2) use clinical judgment to select the type of moist wound dressing for the ulcer; (3) choose a dressing that will keep the surrounding skin intact; (4) choose a dressing that controls exudate but does not desiccate the ulcer bed; and (5) consider caregiver time when selecting a dressing.

Various products are commercially available in wound care, including saline/gauze, films, foams, hydrocolloids, hydrogels, absorptive dressing, calcium alginate, and combinations of these. In the area of biological products, there are growth factors, cultured keratinocytes, dermal skin substitutes, and dermal/epidermal skin substitutes. The cost of biological products is sometimes prohibitive for long-term or multiple applications.

It has recently become recognized that the temperature of a wound bed is an important factor in healing. Sufficient heat may increase the numbers and activity of fibroblasts to counteract the inhibitory effect of chronic wound fluid, to increase profusion to the wound bed and to decrease bacterial count. Sufficient heat increases blood flow to the wound bed and increases oxygen partial pressure.

The recognition that sufficient heat promotes wound healing has not translated well into a broad array of effective commercial products. Primitive devices such as heat lamps have been tried, but have produced destructive drying or even burning. Physicians now commonly believe that heat therapy along with other intervention treatments should be reserved for cases where the natural healing process demonstrates failure. With regard to the associated problem of chronic wound infection, physicians usually utilize ordinary antibiotic treatments. This too is fraught with risk, for vascular antibiotic delivery is ineffective in peripheral vascular-impaired patients.

A series of patents to Augustine Medical, Inc. are directed toward wound heaters, including U.S. Pat. Nos. 6,217,535B1; 6,213,966B1; 6,113,561; 6,095,992; 6,080,189; 6,071,304; 6,071,254; 6,045,518; 6,254,557B1; 6,248,084B1; 6,241,697B1; 6,213,965B1; 6,010,527; 5,986,163; 5,964,723; 5,964,721; 5,961,480; 5,947,914; and 5,817,145. Some of these patents describe systems for controlling the climate of a wound bed using heaters or humidity regulating devices.

Heaters are generally of the electrical resistance type, although it has been recognized that other types of heaters might be usable, such as chemical heaters. It has also been recognized that a substitute for a heater is a passive insulator to maintain body temperature at the wound site by lessening the escape of heat to the surroundings.

A difficulty with active heaters, whether of the electrical resistance type, the chemical type, or otherwise, is temperature regulation. An insufficient temperature fails to achieve the beneficial therapeutic effects for which the system is designed. An excessive temperature can reduce fibroblast activity or even burn tissue. Moreover, temperature fluctuations can impair healing even if both the low end and the high end of the range in which the fluctuations occur are within the therapeutically beneficial temperatures.

The temperature regulator typically used in the few existing devices that include heating elements is usually a thermostat. The heating element is activated or inactivated as the temperature rises or falls in reference to a set point or range. The problem with such thermostats, apart from issues of reliability and cost, is that they are not perfectly sensitive. The wound temperature rises to some point beyond the setting before the heater is turned off, or, even if the heater turns off precisely when the wound temperature rises to the setting, the heater continues to radiate until the thermal absorption elements in it dissipate the remnant heat. Conversely, the wound temperature falls to some point lower than the setting before the heater is turned back on. Thus, unless the thermostat and heater operate with a degree of precision that is impossible except in a theoretical model, the arrangement results in not an exact temperature at the set level but a temperature range surrounding the set level. In practice, this range is at least a few degrees and maybe as much as 5° to 10° F.

It can be appreciated that there is a need for a wound dressing that applies heat to a peripheral wound or other wound and maintains the wound at a constant or very near-constant temperature. Preferably, such a dressing would also control the humidity of the wound by adding moisture to dry sites and removing moisture from exudated sites.

### SUMMARY OF THE INVENTION

The present invention includes a heater to apply heat to a peripheral or other wound site. The heater in a preferred embodiment is an exothermic compound such as an oxidizing metal powder mixed with salts and carbon to regulate the oxygenation reaction. These materials are packaged in an airtight impermeable package that is exposed to air to begin the oxidation heating at the time of use.

The temperature of the wound site is modulated and maintained with a phase-change material. Phase-change materials are those that pass between liquid and solid phases or gas and liquid phases (or, in some cases or circumstances, between gas and solid phases) with a large absorption or release of energy. A familiar phase-change material is water, which absorbs large quantities of energy as it passes from gaseous to liquid at 100° C. and again as it passes from liquid to solid at 0° C.

The particular phase-change material used in the present application must undergo a phase-change at the temperature desired for the wound site, namely at or slightly above a normal body temperature of about 39° C. Such phase-change materials include salt hydrates, paraffin and non-paraffin organics. Paraffins are particularly but not exclusively appropriate because their phase-change temperature is widely and precisely controllable. They present little significant health or safety concerns if properly used, and they can be microencapsulated for ease of packaging into dressing materials. The exothermic compounds can be pelletized for inclusion within the dressing material itself. The phase-change materials can similarly be pelletized and similarly embedded in or attached to the dressing material.

This intermixed arrangement results in very close control of the dressing material. The exothermic compound produces heat. To the extent that heat is in excess of the heat needed to maintain the desired temperature at the wound site, the excess heat is absorbed by the phase-change material changing from one phase to another. By choosing phase-change materials that have a particular phase-change temperature, the wound temperature can be controlled very precisely.

The dressing may also include humidity controlling material such as absorptive dressing for a high exudating wound or moisture-producing or moisture-retaining gel for a low exudating wound. The PCM in small encapsulated form is added to the gels to make a uniform mixture. The gel is used to hold the PCM capsule in suspension and to provide moisture control as per current therapeutic practice. Exothermic materials if used are incorporated into the gel/PCM mixture to generate heat when needed.

### BRIEF DESCRIPTION OF THE DRESSINGS

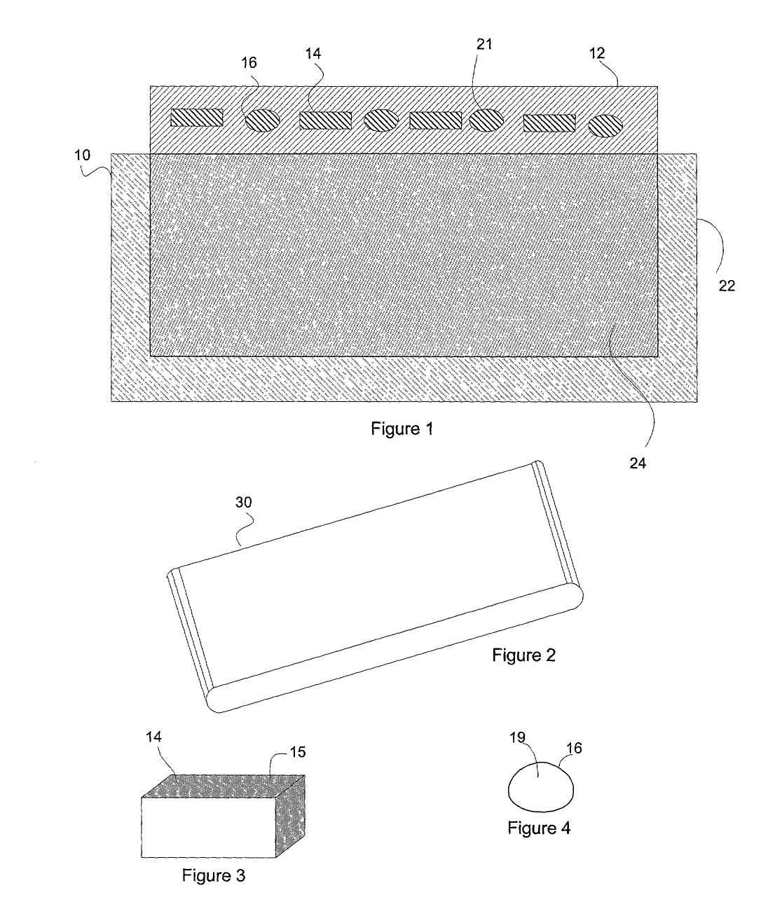

FIG. 1 shows a schematic cross sectional view of a wound dressing in accordance with the present invention.

FIG. 2 shows the wound dressing of a preferred embodiment packaged in a container.

FIG. 3 shows a pelletized exothermic compound in accordance with the present invention.

FIG. 4 shows a pelletized phase-change material in accordance with the present invention.

### DETAILED DESCRIPTION OF A PREFERRED EMBODIMENT

A schematic view of a wound dressing 10 in accordance with a preferred embodiment of the present invention is shown in FIG. 1. The wound dressing 10 includes a substrate 12 which is impregnated with pelletized exothermic compounds 14 and pelletized phase-change materials 16.

The dressing 10 may be made of any suitable dressing material, such as woven natural textiles, nonwoven natural or artificial textiles, or light weight foams. The PCM 16 and exothermic compounds pellets 14 may be attached to the dressing pellets 10 by applying an adhesive coating on the pellets 16 and 14 or by incorporating the pellets 16 into a gel 17 spread or distributed in the dressing 10. In a preferred embodiment, the pellets are approximately 25 microns in diameter.

It should be appreciated that the carrier for the exothermic compounds and for the phase-change materials could be something other than the dressing 10. For example, the exothermic compound and phase-change materials could be attached to one or more sides of the dressing or layered within the dressing. It should also be appreciated that the exothermic compound and phase-change material could be of a shape other than pellets, depending in part on the arrangement with the dressing. For example, they could be granulated, powdered or in sheets, gels or other bulk configurations.

The exothermic compound in a preferred embodiment is an energy source and an oxidizer in which the oxidation reaction is activated by bringing both components into contact in the presence of oxygen. This is essentially an electrolytic reaction involving anode, cathode, electrolytic solution, catalyst and oxidizer. Iron powder or filings is an acceptable anode for the exothermic oxidation of iron. The cathode may be activated carbon. Appropriate catalysts include halogen salts such as calcium chloride. The electrolytic solution may be water.

More specifically, the iron powder or filings may be any metal alloy powder containing elemental iron. A medium with high surface area for the oxidation reaction is desired, such as powder of over 100 mesh. Ideal mesh sizes are between 100 and 400. The reactivity of the iron depends also on the impurities in it.

The cathode of activated carbon should be a highly surface-active material such as carbon black, active carbon or wood charcoal. Active carbon is very porous in its inner structure, thus providing good water-retention properties. It therefore serves as a material to hold the electrolyte water and also absorbs excess water that is evaporated by the heat. Since water is needed as an electrolyte; if it is lost, the reaction would stop or be significantly reduced. The cathode material of activated carbon, non-activated carbon and mixtures thereof comprise 3% to 25% of the exothermic compound by weight in a preferred embodiment.

The catalysts of metal salts promote the electrolytic reaction by providing electrical conduction between the anode and cathode to sustain the reaction. Useful metal salts for this purpose include sulfates such as ferric sulfate, potassium sulfate, sodium sulfate, manganese sulfate, magnesium sulfate; and chlorides such as cupric chloride potassium chloride, sodium chloride (table salt) and calcium chloride. It is important that the salts not be hydroscopic (i.e., attracting water), as such salts tend to make hydrates by inhibiting the escape of water vapor that occurs during oxidation. The preferred metal salts during are sodium chloride, cupric chloride, and mixtures thereof.

The preferred electrolyte is water. Typical quantities of water are 1% to 40% of the total mixture by weight. The most convenient oxygen source for the oxidation reaction is air, although other sources may be used such as pure oxygen and materials with bound oxygen releasable upon chemical reaction.

Referring again to FIG. 1, the substrate 12 of the dressing 10 may be backed by a frame 22 which holds a sponge 24. The sponge 24 holds moisture for the purpose of providing re-moisturizing the substrate as moisture evaporates or draws from the substrate. Such moisture both serves as the electrolyte and serves to moisturize the wound bed. The frame 22 can be fabricated from any impermeable rigid or flexible material, such as plastic. The sponge can be any biologically inert foam-like material that will absorb moisture, of which many are known in the field of medicine. It should be appreciated that the drawing of FIG. 1 is not necessarily to scale; the frame, sponge and other elements may be thicker, or especially thinner in relation to the width of the dressing than shown in the drawing. The dressing 10 should be packaged in an airtight container to avoid prematurely starting the exothermic reaction.

The dressing with these materials should be stored in an impermeable and air-tight package. The impermeability of the package is to ensure that the electrolyte does not dry out by migration or evaporation through the package. The air-tightness is to ensure that oxygen from the air does not pass into the package and start the exothermic electrolytic reaction prematurely.

A drawing of a package for the dressing is in FIG. 2. While package 30 as well as dressing 10 can take a variety of forms, the simplest for illustration involve a planar dressing 10 impregnated with the exothermic compounds encased in a package 30. The package 30, as mentioned above, should be both air-tight and impermeable. A foil package is suitable. The package is opened upon use.

The pellets 14 and 16 of the exothermic compound and phase change material are shown in FIGS. 3 and 4, respectively. The pellets 14 and 16 of FIGS. 3 and 4 are shown schematically; it should be appreciated that they may assume a variety of configurations and sizes. On the outer surface of each of the pellets 14 and 16 are adhesives 15 and 19 to adhere the pellets 14 and 16 to the dressing 10 of FIG. 1. As mentioned above, the pellets 14 and 16 may also be incorporated into a gel 21 which is in turn infused into the dressing 10.

The dressing 10 is used by tearing or otherwise opening the package 30 at the site of use and discarding the package 30 material. The dressing 10 at that time receives exposure to oxygen in the air, and the exothermic electrolytic reaction is activated. The oxidation of the anodic metal pellets 14 by an electrolytic reaction with the cathodic activated carbon pellets 16 proceeds through the catalytic electrolyte. This reaction generates heat. The heat is dissipated largely toward the exposed surface of the dressing 10 and into the wound bed of the patient.

In the absence of a system for regulating this heat, the wound bed temperature would become excessively warm to the point where the healing and therapeutic properties would be comprised, or the tissue at the wound bed could even be desiccated or burned. The phase-change materials serve that regulatory function. As heat from the exothermic material builds to increase the temperature of the dressing, the temperature of the phase-change materials similarly increases. The phase-change materials initially are in the solid phase. When the temperature reaches the phase-change material melting point, the phase-change material begins to melt. The additional heat produced by the exothermic materials is absorbed by the phase-change material melting, until all the phase-change material is melted. During the period of phase-change material melting the temperature stays at the melting point. The phase-change material is chosen such that the melting point is the desired temperature for the wound dressing. This is ordinarily at or slightly above normal body temperature of about 39° C. As mentioned above, a phase-change material having such a melting point is paraffin.

The duration of this period when the exothermic reaction is generating heat in excess of the amount necessary to maintain the dressing at a constant temperature equal to the phase-change material melting point, while the phase-change material gradually melts to absorb that excess heat so that the temperature stays at nearly exactly the melting temperature, can be engineered to be quite long. This is done by using an amount and mixture of exothermic material that generates heat over a long period that is only slightly greater than the amount typically needed to maintain the desired temperature. In that manner, there is a minimum of excess heat that needs to be absorbed by the melting of the phase-change material. The phase-change material consequently melts slowly.

What is claimed is:

1. A wound healing device, comprising: a dressing; an exothermic material in thermal communication with the dressing; and a phase-change material in thermal communication with the exothermic material, the phase-change material having a desired phase-change temperature to maintain the dressing at a temperature to promote healing of the wound.

2. The device of claim 1, wherein the phase-change material includes a plurality of substances having differing phase change temperatures.

3. The device of claim 1, wherein the phase-change material includes paraffin and a hydrated salt phase change material.

4. The device of claim 1, wherein said dressing is absorptive.

5. The device of claim 1, wherein the exothermic materials are embedded in said dressing.

6. The device of claim 5, wherein the phase change material is embedded in said dressing.

7. The device of claim 1, further comprising a moisturizing gel on the dressing.

8. The device of claim 7, wherein at least one of the phase change material and the exothermic compound is in said gel.

9. The device of claim 5, wherein the phase-change materials are pelletized.

10. The wound dressing of claim 1, wherein the exothermic materials include an oxidizing metal anode.

11. The wound dressing of claim 10, wherein the exothermic materials include a carbon-containing cathode, a metal salt catalyst and an electrolyte.

12. The wound dressing of claim 9, wherein the moisturizer is a hydrogel.

13. A method for treating a wound in a human, comprising: applying a dressing having an exothermic material and a phase-change material with a phase-change that is at a temperature to promote healing of the wound; releasing heat from said exothermic material; and absorbing that into the phase-change material so as to change the phase of said phase-change material.

14. The method of claim 13, wherein said phase-change material includes a plurality of materials having differing phase-change temperatures.

15. The method of claim 13, further comprising applying a moisturizing gel to the wound.

16. The method of claim 13, wherein said phase-change material includes paraffin.

17. A method of manufacturing a dressing for treating a wound in a human, comprising: forming a plurality of pellets of phase-change material having a phase-change temperature of a desired temperature for healing the wound; embedding the pellets into the dressing.

18. The method of claim 17, further comprising: embedding into the dressing an exothermic material.

19. The method of claim 18, further comprising applying a moisturizing gel to the dressing.
